(19) United States
(12) Patent Application Publication
(10) Pub. No.: US 2019/0046808 A1
(43) Pub. Date: Feb. 14, 2019

(54) ELECTROSTRICTIVE NANOPARTICLE TRANSDUCERS AS RADIO FREQUENCY ALTERNATING ELECTRIC FIELD SUSCEPTORS FOR RAPID HEATING

(71) Applicant: TDA Research, Inc, Wheat Ridge, CO (US)

(72) Inventors: John M. Alford, Lakewood, CO (US); David P. Eisenberg, Denver, CO (US)

(21) Appl. No.: 16/054,825

(22) Filed: Aug. 3, 2018

Related U.S. Application Data
(60) Provisional application No. 62/540,986, filed on Aug. 3, 2017.

(51) Int. Cl. A61N 1/40; A61K 41/00; B82Y 5/00; B82Y 30/00

(52) U.S. Cl. CPC A61N 1/406; B82Y 30/00; B82Y 5/00; A61K 41/0052

(57) ABSTRACT

The invention provides a method for rapid uniform heating of a target material by providing a target material; providing a plurality of electrostrictive nanoparticles contained in the target material; providing a radio frequency alternating electric field that is coupled to the plurality of electrostrictive nanoparticles; and heating the target material.

### CROSS REFERENCE TO RELATED APPLICATIONS

The present application claims the benefit of the provisional application number 62/540,986 filed Aug. 3, 2017 (titled ELECTROSTRICTIVE NANOPARTICLES AS RADIO FREQUENCY SUSCEPTORS FOR RAPID HEATING, by Mike Alford and David P. Eisenberg, attorney docket number 17-4), which is incorporated by reference herein.

### STATEMENT REGARDING FEDERALLY SPONSORED RESEARCH OR DEVELOPMENT

This This invention was made using U.S. government funding through the U.S. Defense Health Agency SBIR Phase I contract #W81XWH-15-C-0103 and SBIR Phase II contract # W81XWH-16-C-0073. The government has certain rights in this invention.

### BACKGROUND

Alternating electromagnetic (EM) fields can be used to warm materials. For example, heat lamps (which uses near infrared (NIR, 214-400 THz) to heat the surface of objects), or microwave ovens (which use microwave EM fields, 300 MHz-300 GHz). An alternative to heating objects with EM fields directly is to use nanoparticle susceptors coupled to the EM field. Instead of relying on the target material to effectively absorb the EM field, the nanoparticle is designed to absorb the EM field, it then transfers its heat to the target material around it. Nanoparticle coupled heating allows the heating to be more targeted than direct EM heating with possible applications in cancer therapy and cryopreservation, among others.

Nanoparticles generally have a particle size, or at least one dimension (i.e. diameter) that is less than 100 nm. Prior art has attempted to use metallic or magnetic nanoparticles for heating applications with NIR (214-400 THz) or radio frequency (RF, 3 Hz-300 MHz) EM fields, where the field is primarily magnetic in nature (i.e. generated using an inductor). Magnetic nanoparticles are referred to as superparamagnetic materials because they generally are small enough to only have a single magnetic domain.

If magnetic nanoparticles are used EM fields can be coupled by the magnetic portion of the electromagnetic field to the magnetic moment of superparamagnetic nanoparticles. A superparamagnetic particle is a magnetic particle that is so small (nanometers) that it contains only one magnetic domain, and with only one domain, the particle magnetizes and demagnetizes to its saturation limit with no hysteresis. Typical magnetite (Fe₃O₄, iron (II, III) oxide) particles will remain superparamagnetic when less than ˜20 nm in size, depending upon their exact structure. These are known as superparamagnetic iron oxide nanoparticles (SPIONs). The heating of the superparamagnetic particle occurs by either rotation of the entire particle to align with the applied field which in turn creates a viscous drag force (Brownian relaxation), or internal rotation or jumping of the magnetic moment of the particle to align with the applied field without physical rotation of the particle (Noel relaxation). These contributions generate the total relaxation at the frequency of the EM field. The dominant mechanism depends upon the magnetic properties of the particle, such as the magnetic core size, hydrodynamic size, magnetization, anisotropy factor, and the solution viscosity, all of which are functions of the temperature. Since the electromagnetic coupling is magnetically based, a solenoid is used to generate the largest possible alternating magnetic field, with typical frequencies in the 10's of kHz to 1 MHz range. However, in an alternating magnetic field, the solenoid also generates an alternating electric field as required by Maxwell's equations. The solenoid will also have a large AC voltage (alternating electric field) across it, due to its impedance and large alternating current. This electric field can also couple to the sample and induce eddy currents, which cause dielectric heating. In human patients, it is this dielectric heating that ultimately limits the applied magnetic field strength and frequency for both MRI and magnetic hyperthermia treatments. Thus, in this type of prior art, alternating electric fields are considered a problem to be avoided.

Magnetic nanoparticle heating can be used for hyperthermia heat generation. A high nanoparticle concentration is needed because the specific absorption rate (SAR) of the iron oxide nanoparticles is not particularly high, only 300-400 W/g-Fe. Generation of the required magnetic field in a limb sized inductor with its associated large inductance and power dissipation of 10's of kW also presents a substantial engineering challenge.

Plasmon Resonance Nanoparticles (metallic) have been used for dielectric heating of tissues with laser induced plasmon resonance generated by near infrared radiation (214-400 THz) capable of penetrating tissues [Qin and Bischof (2012)]. When monochromatic light impinges on gold (metallic) nanoparticles with the appropriate dimension, the electron cloud in the particle resonates with the light's electric field, similar to a low frequency electric field in a conventional metallic cavity, creating a substantially enhanced absorption that is orders of magnitude larger than conventional organic molecules. The absorbed light is dissipated as heat. Tissues have several optical “windows” with low absorption, and the lowest absorbance region is near 800 nm (NIR); this allows useful access to tissue depths of ˜5-10 mm. Gold nanoparticles or shells tailored to absorb at this wavelength can be efficient susceptors, and are more effective at heating than magnetic nanoparticles for the same number of particles. This makes them attractive for certain applications where the target tissue is easily reachable: however; plasmon resonance heating is not suitable for bulky targets.

Radio frequency (3 Hz-300 MHz) electric fields have been used in attempts to heat with metallic nanoparticles. Electric fields in the low RF to 100's of MHz range uniformly penetrate tissues similar to the magnetic fields used for inductive heating, and the use of nanoparticles as susceptors to generate heat using high voltage electric fields in this range has been suggested: however; these suggestions have only been directed at metallic nanoparticles. These attempts have also been met with poor or inoperative results.

Initial studies of spherical Au nanoparticles (Moran et al., 2009) reported a very high specific absorption rate (SAR) that substantially exceeded the SAR of magnetic nanoparticles by 2-3 orders of magnitude. This result was erroneous as explained by Hanson et al. (2011), who showed that Joule (resistive) heating due to internal conduction in a polarizing spherical nanoparticle could not produce the required heating rate due to the extremely high conductivity of gold. More recent experiments (Corr et al., 2012) using highly purified spherical gold nanoparticles confirmed parts of this theoretical analysis, as the purified samples did not heat efficiently. This suggests that the previous, anomalously high result was due to Joule heating from ions in the buffer solution of the original samples rather than the gold nanoparticles. The prior art demonstrates the unpredictable and not fully understood nature of how nanoparticles can interact with EM fields to generate heat.

There is interest in heating tissue for thermal cancer therapy, or killing cancer by warming the cancer cells. Warming the cells mildly (42-43° C.) is known as hyperthermic therapy or warming them even more (>50° C.) is known as thermal ablation. Both of these approaches can be considerably cheaper and less invasive than surgery and are referred to as cancer thermal therapy. They also tend to have far milder side effects than other non-surgical approaches such as chemotherapy or radiation therapy. When pursuing cancer thermal therapy, it is important that all of the cancerous tissue is treated, while minimizing collateral damage to healthy tissue. To achieve this, the tissue can be implanted with nanoparticles, then exposed to an EM field in a frequency that the tissue doesn't absorb. This way, the nanoparticles in the cancerous tissue with absorb the EM field and heat the tissue, while the healthy tissue without the nanoparticles won't absorb the EM field, and hence, won't be heated.

This is the approach employed when SPIONs are used as the nanoparticle susceptor. SPIONs exhibit fairly low cytotoxicity and are activated using low frequency RF (0.1 to 1 MHz), which can easily penetrate through the tissue to only activate the SPIONs. Their main drawback is that their heating effectiveness, known as specific absorption rate (SAR) is fairly low (only 100s of W/g-Fe). This means that thermal therapies require a high concentration of SPIONs to be effective. This high concentration can be difficult to achieve in real world settings, additionally, high concentrations are more likely to cause problems with toxicity.

Thermal therapy with plasmon resonance nanoparticles (metallic nanoparticles) has the opposite problems as the SPIONs. The gold plasmon particles couple with NIR instead of RF, so the penetration depth is very limited. This means that cancerous cells deep in the body are either inaccessible or have to be irradiated with fiber optics. However, the gold plasmon particles produce orders of magnitude more heat per mass of particles. This means that they can be effective, even in very low doses.

In addition to direct thermal therapy, nanoparticles can be used to selectively release drugs for cancer therapy. The nanoparticles can be functionalized so that they preferentially seek cancer cells. Cancer drugs can then be attached to the nanoparticles with bonds that break when the nanoparticles are activated via electromagnetic field. Improving the nanoparticle's heating rate in a given field can improve the efficiency of the cancer drug delivery

Another application of heating tissue is in thawing cryopreserved tissues. There are two primary problems associated with cryogenically preserving tissues, the freezing step and the thawing step. In both cases any phase change that forms crystalline ice will causes significant tissue damage (frostbite is a good example, where tissue necrosis is common). To solve the freezing problem, tissues are perfused with cryoprotective agents (CPAs). CPAs act as freezing depressants, and exhibit exponentially increasing viscosity with decreasing temperature. With high enough CPA concentration and a fast enough cooling rate, the water goes from the liquid phase directly to a glassy vitreous state without forming crystalline ice. Unfortunately, thawing is actually harder than cooling. Firstly, the warming rate necessary to prevent crystal formation (known as the critical warming rate) tends to be ˜10× higher than the critical cooling rate. Additionally, due to the viscoelastic nature of the CPAs and tissues perfused with the CPAs, the mechanical stress due to non uniform warming is generally much higher than the mechanical stress due to temperature gradients during cooling [Eisenberg 2014, Steif 2007] and can lead to mechanical damage which renders the tissue non viable post thawing. There have been attempts in the past to use EM fields to directly warm cryopreserved tissues, but these were largely unsuccessful due to limited penetration depth, spatial non-uniformity due to high frequencies [Cooper 1981, Gordon 1982], and temperature dependent absorptivity leading to thermal runaway [Evans 2000, Robinson 2002].

Nanoparticles coupled with EM can increase the warming rate, have a much larger penetration depth and almost no spatial non-uniformity (if they are designed to work with lower frequency EM), and do not have temperature dependent absorptivity, eliminating the possibility for thermal runaway. For instance, the 100s KHz EM fields used with SPIONs can easily penetrate deep into tissue and doesn't have any problems with non-uniformity of thermal runaway [Bischoff 2014]. It's also been shown theoretically that tissues thawed with SPIONs coupled with magnetic RF can have drastically lower mechanical stress than tissues warmed via a hot water bath [Eisenberg 2015]. However, these SPION nanoparticles still have a low SAR (300 W/g) and, thus, low heating rates (less than 1° C./sec, even at very high nanoparticle loading levels).

The individual prior art teachings (regardless of high or low frequency EM fields use) each suffer from at least one limitation. High frequency electromagnetic radiation has been used for cancer thermal therapy and thawing cryopreserved tissue. These frequencies are in the 100's or 1000's of MHz. This has three main drawbacks: (1) high frequency radiation does not penetrate very far into biological tissue. This makes it hard to treat cancers within the body. For cryopreservation, this causes large thermal gradients within the tissue (increasing mechanical stress and the likelihood of ice crystal formation), or there has to be strict size limitations on what could be rewarmed this way. (2) High frequency translates to short wavelengths, the nodes of the wavelengths are cold spots while the anti-nodes are hot spots. (3) For cryopreservation, due to the temperature dependent absorptivity of electromagnetic radiation in tissue, where warmer tissue has a higher absorptivity, small deviations in the temperature field due to the hot and cold spots can snowball into “thermal runaway” where some sections are far too warm while other sections are far too cold.

Additionally, lower frequency, electro-magnetic (RF) fields (0.1's of MHz to 10's of MHz) have been used, coupled with super paramagnetic iron oxide nanoparticles (SPIONs) for hyperthermic cancer therapy, and more recently, for uniformly thawing cryopreserved tissue. This lower frequency of electromagnetic radiation can easily pass through biological tissue without attenuation, eliminating the 17-4B problem of limited penetration depth seen at higher frequencies. In fact, the RF radiation barely interacts with the tissue at all, instead, it induces Brownian and Néelian relaxation mechanisms in the nanoparticles, generating heat which is then dissipated to the surrounding tissue. Also, with the lower frequency magnetic field, the wavelengths are long enough that there is little effect due to the spacing of nodes and antinodes, eliminating the concern with hot spots and thermal runaway. The main drawback of using low frequency RF and SPIONs is that they are not very effective heaters (SAR values are typically in the 100's to low 1000's of W/g-Fe), requiring very high nanoparticle loading of 5-10 mg/ml in order to induce the desired heating rate.

There has also been work using metallic (gold) nanoparticles coupled with NIR to cause plasmon resonance in the particles. These are more effective heaters than the SPIONs, meaning that they can be used in much lower concentrations. However, the penetration depth of NIR in tissue is very small (5-10 mm). This means that cancer therapy can only be conducted near the surface, or fiber optics need to be inserted into the body, making the process more invasive and more technically difficult. For thawing cryopreserved tissues, the limited penetration depth means that only very small samples can be rewarmed using this method.

### BRIEF SUMMARY OF THE INVENTION

The present invention teaches that unlike the inferior metallic, magnetic or superparamanegtic nanoparticles, the class of electrostrictive nanoparticles can respond very strongly to radio frequency electric fields and heat surrounding materials. Without wishing to be being bound by theory, electrostrictive nanoparticles primarily respond to the electric field in radio frequency (RF) electromagnetic (EM) radiation to uniformly generate heat, and could be used to generate heat needed for cancer thermal therapy or to rewarm cryogenically preserved tissues and cells, solving the limitations of the prior art. Electrostrictive nanoparticles experience electron motion (polarization) when coupled with alternating electric fields, causing them to elongate in the direction of the electric field, and this cyclic elongation may generate heat. In addition, certain types of electrostrictive materials are also piezoelectric. This means that they polarize and deform in response to an electric potential, but the polarization and the deformation is not necessarily aligned with the excitation electric field. This cyclic deformation due to radio frequency alternating electric fields may also be partially responsible for generating heat. If the piezoelectric particles' polarization is not aligned with the applied electric field, the nanoparticle then might rotate in order to align with the electric field, generating heat via viscous dissipation. The present invention teaches the use of RF frequency alternating electric fields, with electrostrictive/piezoelectric nanoparticles that respond to the electric field. In this situation, less power and a lower nanoparticle concentration is required to heat cancer cells and cryogenically preserved tissues compared to magnetic induction heating. Other solids (beyond tissue) or liquids can be heated using the methods of the present invention.

In addition, certain types of piezoelectric materials are also ferroelectric. Ferroelectric materials exhibit a spontaneous electric polarization (they are named after ferromagnetic materials, which exhibit spontaneous magnetic polarization). Single domain ferroelectric nanoparticles may also generate significant heat when subjected to radiofrequency alternating electric fields.

The present invention provides a method for rapid uniform heating of a material, the method comprising: providing a target material; providing a plurality of electrostrictive nanoparticles contained in the target material; providing a radio frequency alternating electric field that is coupled to the plurality of electrostrictive nanoparticles; and heating the target material. Alternatively, the plurality of electrostrictive nanoparticles are piezoelectric nanoparticles, more preferably CeO₂ or ZnO nanoparticles. Alternatively, the plurality of electrostrictive nanoparticles are ferroelectric nanoparticles

In other embodiments, the plurality of electrostrictive nanoparticles have an average particle size less than 1 μm; the radio frequency alternating electric field is from 1 to 100 MHz; the radio frequency alternating electric field is from 30 to 50 MHz; or the radio frequency alternating electric field strength is 315 kV/m.

In a preferred embodiment, the plurality of electrostrictive nanoparticles have an SAR of at least 1,000 Watts per gram, more preferably at least 18,000 Watts per gram, more preferably still at least 20,000 Watts per gram.

In an embodiment, the target matrix is either: water, living tissue or cryopreserved tissue.

In another embodiment the plurality of electrostrictive nanoparticles are uniformly dispersed in the target material.

In an alternative method that heating method further comprises the step of: heating the target material by at least of 1.0° C. per second; or more preferably, heating the target material by at least 3.5° C. per second.

In a further embodiment, the method further comprises: providing a first electrode and a second electrode; providing a device for generating a radio frequency alternating electric field; wherein, the target material is placed between the first electrode and the second electrode, wherein the first electrode, the target material and the second electrode are assembled to form a capacitor cell; wherein, the device for generating a radio frequency alternating electric field is operably connected to the capacitor cell; and wherein, an alternating electric field is exposed to the target material.

In an optional embodiment, the capacitor cell further comprises an electrically insulating housing for the target material., which can be made from either Teflon®, Rexolite®, Kapton®, or combinations thereof.

The present invention teaches that piezoelectric nanoparticles can be used for heating applications, including for warming biological tissue. The SPIONs described in the Background rely mostly on the magnetic part of the electromagnetic field (generated via an inductor) while the piezoelectric nanoparticles of the present invention rely on the electric part of the electromagnetic field (generated via a capacitor). The piezoelectric nanoparticles that are the focus of this invention are an improvement over the SPIONs. The lowest performing piezoelectric nanoparticles that we have tested, CeO₂, demonstrated SAR values in the high 1000's of W/g-CeO₂ while the preferred particle, ZnO, demonstrated an SAR of >23,000 W/g-ZnO (60-80 times better than comparable SPIONs used for thawing of cryopreserved tissue). These high heating rates and SAR values were found at a relatively modest loading of 1 mg/ml (instead of the 5-10 mg/ml of SPIONs needed). 0.1 mg/ml, 1 mg/ml and 10 mg/ml), but still found significant heating even at only 0.1 mg/ml ZnO.

Maxwell's equations dictate that electric and magnetic fields are always generated together when they are oscillating (i.e. electromagnetic fields). Solenoids accentuate the magnetic field within the area of the solenoid while capacitors accentuate the electric field between the capacitor plates. The SPION nanoparticles are activated using magnetic fields in a solenoid. Whereas, the electrostrictive nanoparticles of the present invention are activated using an electric field in a capacitor. This is a critical difference between methods of the prior art and the present invention.

### BRIEF DESCRIPTION OF THE DRAWINGS

FIG. 1. Capacitor cell.

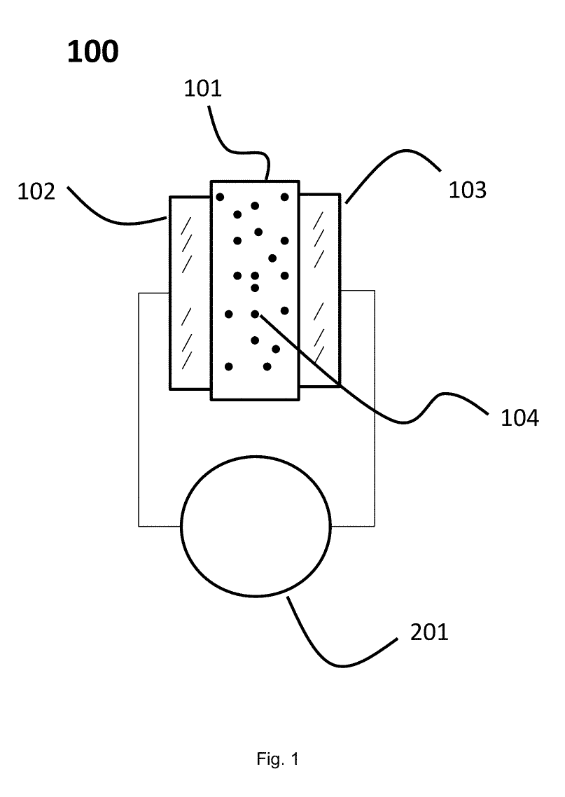

FIG. 2. Alternative capacitor cell.

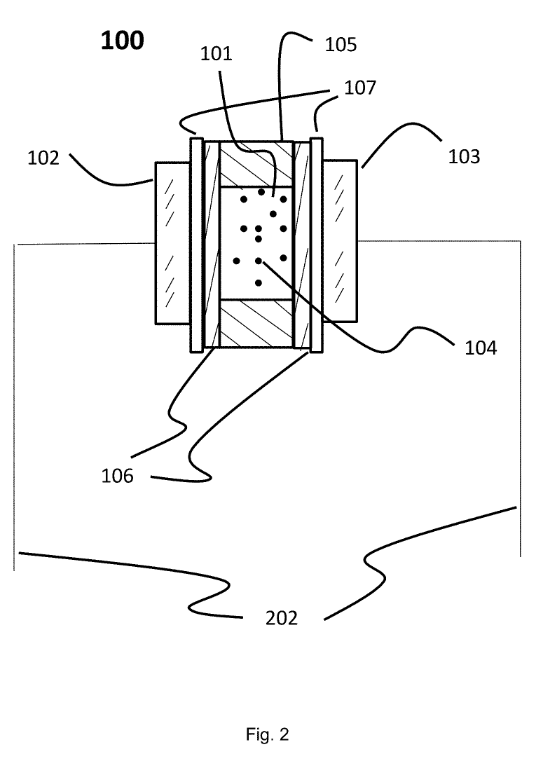

FIG. 3. Resonant circuit RF heating device schematic.

FIG. 4. Heating of nanoparticles dispersed in deionized water at a nanoparticle concentration of 1 mg/mL (⅛-inch cell and at 10 Watts)

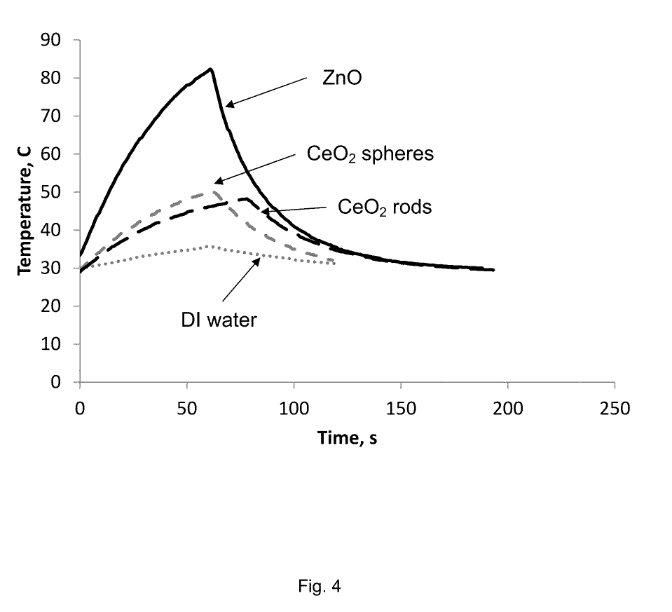

FIG. 5. Heating of ZnO nanoparticles with a concentration of 1 mg/mL in deionized water in a ⅛-inch cell.

FIG. 6. Equations for calculating SAR.

FIG. 7. SAR for nanoparticles tested as a function of power supplied to the resonant circuit.

FIG. 8. Heating of ZnO vs. the supernatant after particles have been partially removed by centrifugation.

FIG. 9. Heating of ZnO nanoparticels at three different concentrations in deionized water.

FIG. 10. Capacitor models including dielectric losses.

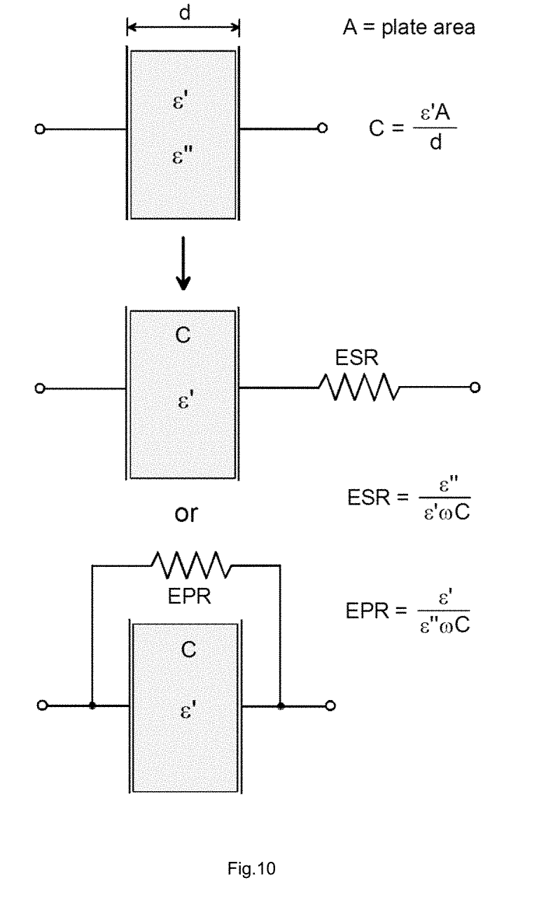

FIG. 11. Calculated voltage, current, and power at resonance.

### DETAILED DESCRIPTION OF THE INVENTION

The present invention teaches heating a target material with high heating rates (>3.5° C./sec) using piezoelectric nanoparticles (CeO₂ and ZnO) within a RF alternating electric field. In a preferred embodiment the RF field is 40 to 50 MHz with a strength of ˜315 kV/m. Considering the observed nanoparticle loading, sample size, and heat loss observed in the examples below, this translates to a specific absorption rate (SAR) of >23,000 W/g of nanoparticles, which is orders of magnitude higher than the SAR reported for super paramagnetic iron oxide nanoparticles. The preferred nanoparticles of the present invention are ZnO, although other electrostrictive, and preferably piezoelectric nanoparticles can be used. Without wishing to be bound by theory, the heating mechanism taught by this invention is due to the cyclic deformation that piezoelectric nanoparticles undergo when subjected to alternating electric fields. Thus, any piezoelectric nanoparticle stimulated at RF frequencies can be substituted and used in this heating method. Due to the ability to perfuse nanoparticles throughout a target material and the ability for RF to uniformly penetrate biological samples, this method can be used for uniformly generating heat within biological samples. This method can be used for applications in thawing cryopreserved tissue, or alternatively for cancer thermal therapy or thermal ablation.

The word, electrostriction, means a property (found in dielectric materials) caused by a slight displacement of ions in the crystal lattice upon being exposed to an external electric field. Positive ions will be displaced in the direction of the field, while negative ions will be displaced in the opposite direction. This displacement will accumulate throughout the bulk material and result in an overall strain (elongation) in the direction of the field. The thickness will be reduced in the orthogonal directions characterized by Poisson's ratio. All insulating materials consisting of more than one type of atom will be ionic to some extent due to the difference of electronegativity of the atoms, and therefore exhibit electrostriction.

The word, electrostrictive, or the phrase, electrostricitve material(s), means a material that possesses the electrostriction property.

The term, piezoelectric effect, mean a result from the linear electromechanical interaction between the mechanical and electrical states in a crystalline material with no inversion symmetry. The piezoelectric effect is a reversible process: materials exhibiting the piezoelectric effect (the internal generation of electrical charge resulting from an applied mechanical force) also exhibit the reverse piezoelectric effect, the internal generation of a mechanical strain resulting from an applied electrical field.

The word, piezoelectric, means a material that exhibits the piezoelectric effect.

The word, ferroelectric, means a material that exhibits a spontaneous nonzero polarization, even when the applied electric field is zero. Ferroelectric materials are a sub-class of piezoelectric materials.

The word, nanoparticle, means a particle smaller than 1,000 nanometers (1 micron), or a particle having at least one dimension smaller than 1,000 nanometers.

The term, target material, means a solid, liquid, semi-solid, or biological material (i.e. tissue) that is to be heated using the method of this invention.

The acronym, SAR, means “Specific Adsorption Rate” and can be expressed in units of Watts per gram of the nanoparticles used in the heating method of the present invention.

The trademark, Telfon®, is a synthetic fluoropolymer of tetrafluoroethylene.

The Trademark, Rexolite®, is a cross-linked polystyrene, microwave plastic manufactured by C-Lec Plastics Inc. This material has stable electrical properties into the Giga-hertz frequency range.

The Trademark, Kapton®, is a polyimide film developed by DuPont that remains stable across a wide range of temperatures, from −269 to +400° C.

In the specification of this invention the term electrostriction is used. Electrostriction is exhibited by dielectric materials. When they are subjected to an electric field, positive ions will congregate in the direction of the field while negative ions while congregate in the opposite direction. The cumulative effect of this ion movement is a positive strain in the material (i.e. elongation) in the direction of the field along with contraction perpendicular to the field due to the Poisson effect.

A subclass of electrostrictive materials exhibit the piezoelectric effect. The piezoelectric effect is when a mechanical deformation causes the material to have a voltage across it. Piezoelectric materials also exhibit the inverse piezoelectric effect, where materials subjected to an electric field deform mechanically. The piezoelectric effect (and the inverse piezoelectric effect) is due to asymmetry in the distribution of charges within the crystal structure. When the structure is deformed along certain axes, the asymmetry causes the charges to become unbalanced, leading to a voltage. Similarly, when a voltage is applied, the asymmetry causes the charges to attract or repel, leading to mechanical deformation.

Piezoelectrics are a type of dielectric. Piezoelectrics generate an electric field when deformed due to an applied external force (the piezoelectric effect), and deform when subjected to an external electric field (the inverse piezoelectric effect). An alternating electric field causes cyclic deformation of piezoelectric nanoparticles. This cyclic deformation dissipates energy in the form of heat. The method of the present invention teaches the use of piezoelectric nanoparticles with RF alternating electric fields for heating of a target material.

In an embodiment RF alternating electric fields heat a target material, which has been perfused with electrostrictive nanoparticles. RF electric fields can penetrate body tissue with minimal attenuation. Therefore, by using the nanoparticles as nano-heaters, the RF alternating electric field can volumetrically heat tissue samples. Additionally, at the frequency range (10-90 of MHz), the wavelength is >3 m, so there is no problem with hot spots or cold spots allowing for selective, uniform, volumetric heating of tissue.

The invention teaches the use of a capacitor cell 100, in which the target material 101 is infused with electrostricitve and preferably piezoelectric, nanoparticles 104 and positioned between a first electrode 102 and a second electrode 103, forming a capacitor. The capacitor cell 100 is operably connected to a device or generating a radio frequency alternating electric field 201. The first and second electrodes can be made from copper, gold, silver, or other suitable conductor.

Optionally, the capacitor cell 100 may additionally have a sample housing 105 made from Rexolite® or suitable equivalents. The housing may be sealed on the ends by an adhesive insulating material 106, for example Kapton®. The capactor cell may have thermally insulating sheets 107, made from Teflon® or other insulating material. This optional capacitor cell is operably attached to a device for generating a radio frequency alternating electric field with electrical connections 202 attached to the first electrode 102 and the second electrode 103.

In one embodiment, a device for executing the step of generating a radio frequency alternating electric field comprises an RF generator 300, a reflected power meter 301, a signal wire 302, a ground 301, a matching capacitor 304, and inductor 305, a variable capacitor 306, and is operably connected to the capacitor cell 100. In another embodiment, the reflected signal meter 301 is removed and the RF generator 300 directly attaches to the signal and ground.

Experiments can be used to measure the heating effect within an RF field. For example, in the following experiments a variety of dielectric nanoparticles were tested in a variety of concentrations dispersed in de-ionized water. A fiberoptic temperature sensor was used to detect heating, along with a resonant circuit which could generate approximately 1000 V across the plates of a capacitor. These tests used a capacitor spacing of 0.4 inches (for an E field strength of 98 kV/m). Some dielectric nanoparticle types (Fe₂O₃, Si, and BaTiO₃) showed no signs of heating. However, the piezoelectric nanoparticles, ZnO and CeO₂, demonstrated measurable heating over the span of a few minutes. We also reduced the plate spacing to 0.25 inches and then 0.125 inches (for E field strengths of 158 kV/m and 315 kV/m respectively). This drastically improved the heating rates of these piezoelectric nanoparticles. In a preferred embodiment, ZnO is used to give a heating rate of ˜3.5° C./s, at a nanoparticle loading of only 1 mg/ml.

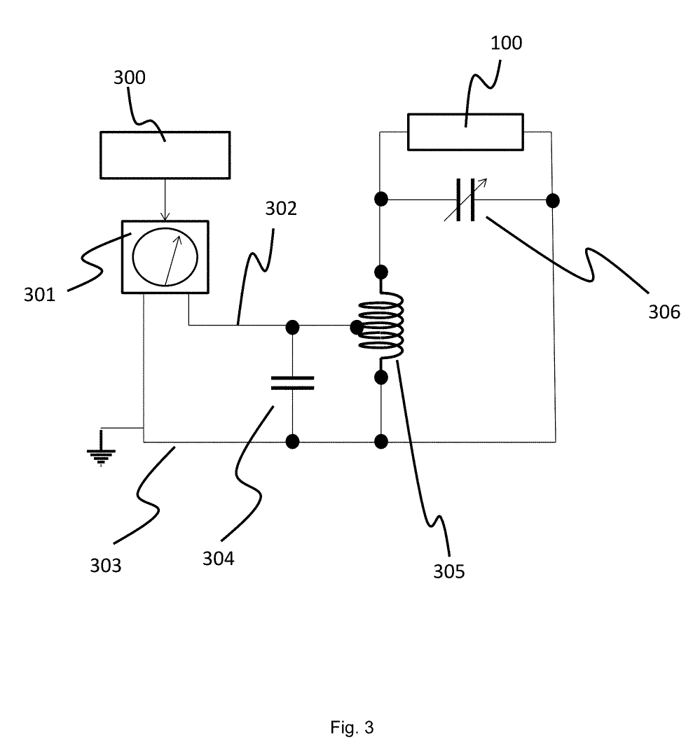

The resonant circuit device used for the above example is shown in FIG. 3. The resonant circuit uses a large (0.31 pH) inductor and tunable vacuum capacitors to generate a high RF voltage on a capacitor designed to fit the nanoparticle sample in an RF compatible cell. The RF signal was supplied by a HP E4421B signal generator coupled to an ENI broadband linear power amplifier with 70 watt output. The forward and reflected power was measured with a BirdRF directional power meter to ensure proper tuning and matching to the 50 Ω amplifier output. Measurements with a 20× voltage divider constructed from a 1 MΩ (2W) resistor showed that the circuit developed approximately 1000V with a 10 W input signal. The 10 watt input corresponds to a voltage of 22.4V into a 50 Ω load, giving a voltage multiplication factor of ˜45. The voltage divider was removed during testing and replaced with a capacitatively coupled 10× scope probe adjusted to give a few hundred millivolt signal so that a relative measure of the voltage could be confirmed for each measurement. For example changing the power from 10 to 20 watts changed the voltage by 1.4 times as expected. High heating samples indicated a slight drop in voltage. The device ran tests at 50 MHz.

Examples of nanoparticle heating: We tested six different types of nanoparticles. Four of them, Fe₃O₄, Si, CeO₂ and ZnO were spherical semiconducting nanoparticles. We also obtained CeO₂ rods and BaTiO₃. These represent an array of compositions, morphologies, and sizes.

All of the experiments were conducted at a frequency of 50 MHz with an electrode spacing of either ¼″ or ⅛″. Experiments were performed at two different power levels, 10 W and 20 W. Voltage on the electrodes was approximately 1000 V at 10 W. Our first set of experiments were performed using DI water in order to get a baseline reading of how much heat will be absorbed from the DI water in RF without the nanoparticles (approximately 0.1 W). All sample suspensions were measured with a NanoTrac (Microtrac Corp.) particle size analyzer and were sonicated for 30 seconds with a Fisher Scientific Model 100 Ultrasonic Dismembrator immediately before the measurement, to evenly disperse the nanoparticles in the target material, and then allowed to equilibrate with the cell before starting the measurement. The deionized water and ZnO sample conductivities were measured with an Orion model 142 benchtop conductivity meter to quantify ionic impurities.

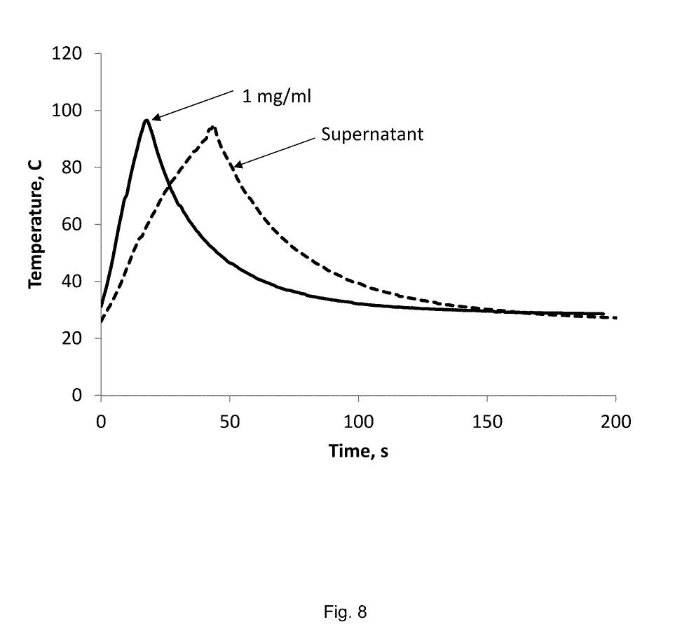

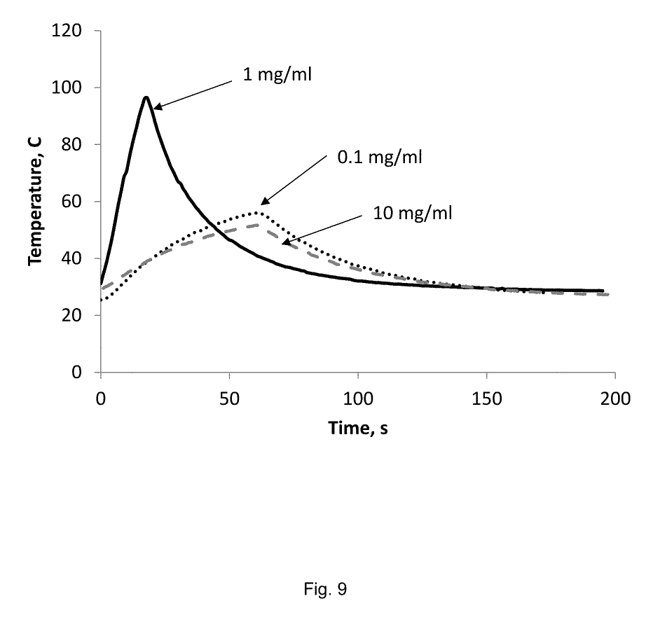

The Fe₃O₄, Si, and BaTiO₃ showed no additional signs of heating when dispersed in DI water. ZnO and CeO₂ however showed significant heating as shown in FIG. 8. As expected, heating increased as electrode spacing decreased and as power was increased. Despite large heat losses to the surroundings, ZnO was heating at a rate of approximately 3.5° C./s under the most favorable conditions as shown in FIG. 9. ZnO and CeO₂ are piezoelectric materials, whereas Fe₃O₄, Si, and BaTiO₃ are not.

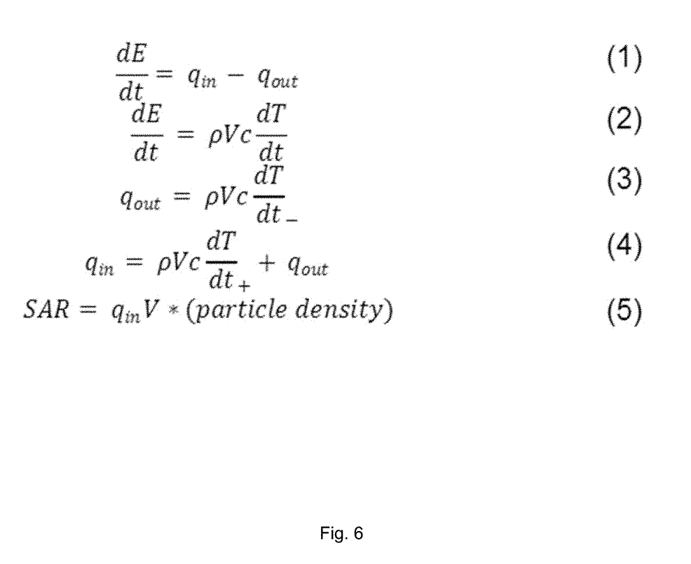

The Specific Adsorption Rate (SAR) was calculated for the nanoparticles by taking into account the heat loss to its surroundings using the equations shown in FIG. 6.

The analysis begins with an energy balance, where the change of energy in the system is equal to the energy going into the system minus the energy leaving, where E=energy, t=time, and q=heat flow. In a thermal system, the change in energy is equal to the thermal mass (ρVc) multiplied by the change in temperature; where, ρ=density, V=volume, and c=specific heat. The heat leaving the system is equal to the thermal mass multiplied by the rate of change of temperature just after the RF device is turned off (denoted by a “−” subscript). Combining equations 1-3, it becomes clear that the heat coming into the system is equal to the slope of the temperature vs. time curve just before the RF is turned off (denoted by a “+” subscript) in addition to the heat leaving the system. The SAR is the heat coming into the system divided by the mass of nanoparticles in the system. This SAR can then be used to compare how effectively a given nanoparticle type heats, independent of nanoparticle concentration.

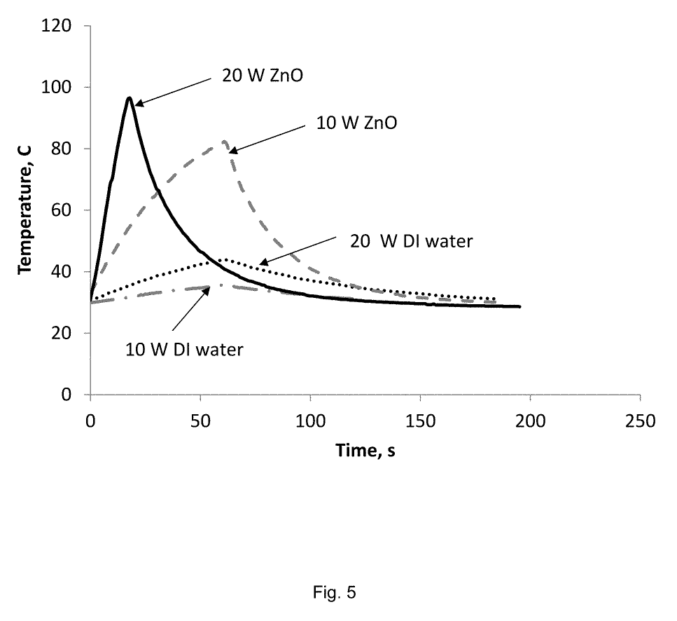

Based on equations 1-5, the SAR was calculated for the CeO₂ spheres, CeO₂ rods, and ZnO nanoparticles. The SAR values are extremely high (FIG. 5). For comparison, the SAR values observed for magnetic Fe₃O₄ nanoparticles (Etheridge 2014) were approximately 300-400 W/g. The SAR value for the ZnO nanoparticles at a 1 mg/ml loading was about 23,000 W/g. This means that ZnO nanoparticles are approximately 60-80 times more efficient at heating than magnetic Fe₃O₄ nanoparticles.

Because of the surprising and very high SAR value that we measured, we carried out additional experiments to confirm these SAR values and verify that the heating was due to the nanoparticles. In the past, groups have attempted Joule heating with alternating electric fields and also reported high SAR values (Moran et al 2009). They attributed the very high SAR values to the nanoparticles. However, upon further experimentation, they realized that the heating was actually due to ionic contaminants in their solution and not due to the nanoparticles. In order to eliminate ionic heating, we carried out two experiments. Since the ions are introduced with the ZnO nanoparticles (as coatings and/or contaminants) we separated and removed some of the nanoparticles using a centrifuge (10,000 g, 30 minutes) while leaving the ions in solution. We then re-tested the solution. The centrifuge was only able to remove some of the nanoparticles. Still, if the heating was due to ions and not the nanoparticles, the heating rate should remain unaffected. Secondly, we increased the nanoparticle (and therefore the potential ionic contamination) concentration in the solution. If the ions were responsible for heating in our system, the heating rate should proportionately increase with increasing the nanoparticle concentration (and the ion concentration).

Based on the data shown in FIG. 8, we see that removing some of the nanoparticles from the solution and leaving in all the ions resulted in a significant decrease in the heating rate (3.6° C./sec vs. 1.57° C./sec). Increasing the nanoparticle concentration from 0.1 mg/ml to 1 mg/ml significantly increased heating rates. However, as the concentration was increased from 1 mg/ml to 10 mg/ml the heating significantly dropped. This is likely due to concentration dependent agglomeration. When the concentration of nanoparticles got too high, the nanoparticles agglomerated and were no longer effective heaters. If the heating were really due to ionic contamination introduced with the nanoparticles, then we should expect the 10 mg/ml solution to heat faster than the 1 mg/ml solution. The increase in ionic concentration was confirmed by conductivity measurements (1 mg/ml=5.5 μS/cm, 10 mg/ml=23 μS/cm). Both of these results confirm that the surprisingly high SAR values that we've calculated are accurate, and not due to contamination by ions.

Discussion of experimental sample purity: Given the contradictory nature of previous studies on RF nanoparticle heating, we considered it imperative to analyze our heating results in detail. Although not wishing to be bound by theory, the following discussion further explains why our observed heating cannot be due to ionic impurities in the samples, and must therefore be due to an intrinsic property of the nanoparticles. To accomplish this, we developed a simulation of the entire resonance circuit including a model of all of the components (inductor and capacitors), the sample cell, and the sample. In this manner, we were able to determine the actual applied voltage, current, and absorbed power for each component including the sample. This was then related to the expected heating rates based upon the known complex dielectric constant (ε′+jε″) of each component. Once this was performed, the measured sample heating rates were compared to the predicted heating rates of both water and ionic solutions using their known dielectric constants combined with formulas relating sample conductivity, σ, to the dielectric loss factor, ε″.

As we will show, the theoretically calculated heating rates were in very good agreement with the measured values for our deionized (DI) water standards. Combining this with the predicted heating rates using the conductivity measurements of our DI water and nanoparticle samples, it becomes apparent that there are not nearly enough ions to explain the observed heating rates. This agrees with the experimental data shown in FIG. 8, where increasing the nanoparticle concentration by a factor of ten actually reduced the heating rate. The only known dielectric losses in the RF region are due to polarization (rotation & vibration of molecular dipoles in the liquid) and the conductivity of ions in the solution. Since the heating cannot be explained by ionic conductivity, it must be due to an intrinsic property of the nanoparticle related to either its polarizability, or as we are presently teaching, a piezoelectric or electrostriction effect (technically, this is still a result of polarization) on the crystal structure of the nanoparticle that causes it to mechanically oscillate in the alternating RF electric field. This vibration then heats the lattice which then transfers that energy to nearby water molecules, heating the sample. If the nanoparticle is a piezoelectric or ferroelectric, and the induced or permanent dipole is not alignedw ith the electric field, then rotation of the particle can also heat the sample. This is a surprising result based on the known shortcomings of the prior art.

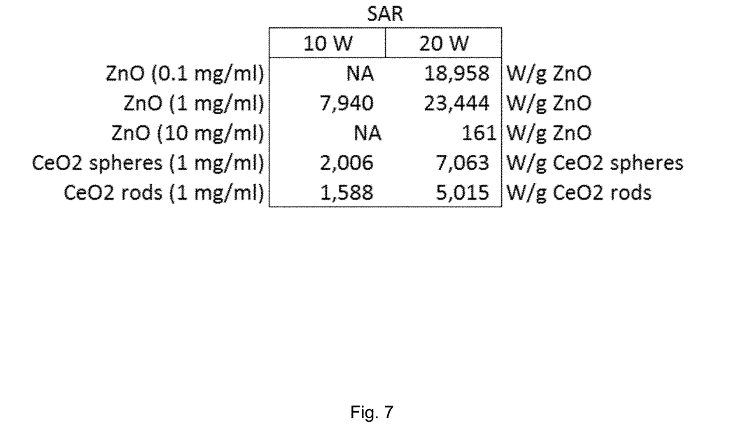

RF Heating Model: To understand how heating in an RF electric field occurs from a macroscopic circuit viewpoint (versus the molecular viewpoint) it is instructive to model the sample as a simple parallel plate capacitor including a lossy dielectric, as shown in FIG. 7. When a voltage is applied to the capacitor plates, an electric field is created across the dielectric material, causing it to polarize. In insulators at radio frequencies, polarization is normally due to reorientation (rotation) of molecular dipoles and separation of charges in the dielectric. The net effect of the polarization is to lower the electric field in the dielectric, since it appears as an electric field opposing the applied field, and to increase the capacitance and energy storage. The induced polarization is proportional to the applied electric field and the permittivity or dielectric constant, ε′. Reorientation of dipoles is almost instantaneous at radio frequencies, but drag forces (due to hydrodynamic forces in a liquid, for example) can cause a slight time delay. The polarization then lags the applied field slightly and the energy loss due the drag force appears as a power loss and internal heating of the dielectric. This loss is specified by a dissipation factor, ε″, which is defined as the imaginary part of the complex dielectric constant, ε=(ε′+jε″). In realistic capacitor models, the effect of ε″ is modeled by either an equivalent series resistance (ESR) or equivalent parallel resistance (EPR) as shown in FIG. 7. The power loss through this resistance corresponds to the dielectric loss, and this formulation now allows us to relate the dielectric loss to conventional circuit (network) elements suitable for modelling. In practice, the power loss in capacitors is commonly specified by the dissipation factor or loss tangent, tan(δ), which equals the ratio of the ESR to the magnitude of the reactive impedance, 1/ωC, and is also equal to the ratio ε″/ε′.

The resonance heating apparatus schematic shown in FIG. 3 was converted to circuit simulation model. All inductors and capacitors were modeled as real devices (i.e., including equivalent series resistances) to permit calculation of all relevant power losses. The inductance and series resistance of the tapped resonance inductor was calculated by measuring its physical dimensions (diameter=25 mm, height=50 mm, and n=5 turns). The inductors on each side of the tap (1 turn and 4 turns) were calculated similarly, and converted into an air-coupled transformer with a k factor. The resonance vacuum capacitor was modeled using a loss tangent of 1.2×10⁻⁴ and computing an ESR at the resonance capacitance. The sample cell was modeled as a parallel plate capacitor based on its geometry and construction materials, Rexolite® (RF grade polystyrene), Kapton® polyimide film, and Teflon® combined with dielectric properties obtained from the manufacturers. The active part of the sample cell (i.e., the surface area of the portion containing the liquid) was placed in parallel with the remainder of the sample cell and also in parallel with the vacuum resonance capacitors. Since the sample cell has thin Teflon® spacers and Kapton® film between it and the electrodes, it was modeled as separate parallel plate capacitors made from each of these materials placed in series.

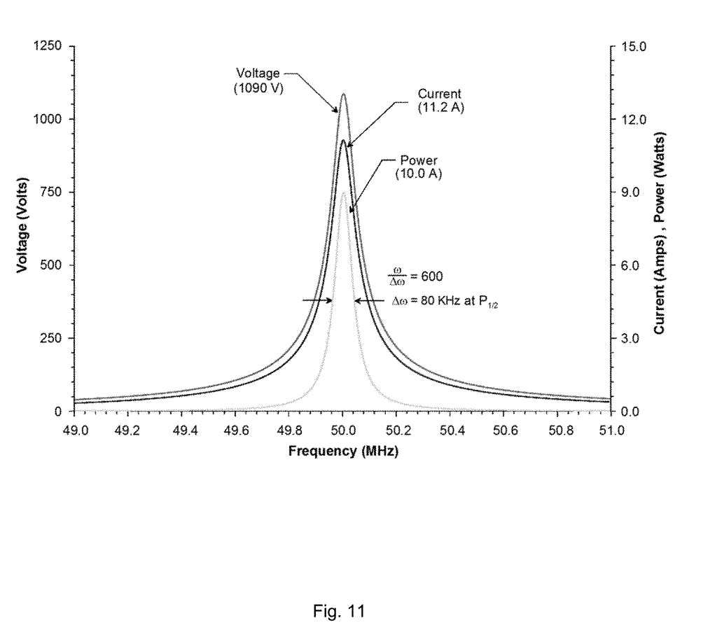

The simulation was run numerous times while tuning to the 50 MHz resonance frequency and 50 Ω input impedance in the same manner as tuning of the actual apparatus. The final simulation results for the voltage, current, and power for 10 watts input as a function frequency are shown in FIG. 11. There is good agreement with the estimated voltage at resonance, ˜1000V. Estimating the loading of the Q from the impedance of the divider, shows that the voltage should drop about 5% when being measured. The estimated change in frequency per change in the resonance capacitance is 700 kHz/pF, showing that just a small, 0.1 pF change in the sample capacitance will shift the frequency by an amount greater than the half width at half power. Dielectric calculations on pure water, shows the relative dielectric constant, ε′/ε₀, changing by −0.28/° C., and for our sample size, the frequency shift will be larger than the half width of the resonance peak for changes on the order of 5° C. at room temperature. As water freezes, the rotational motions become substantially hindered and the relative dielectric constant changes from 87.5 for water at 1° C. to 3.2° C. for ice at −1° C. This corresponds to a capacitance change of 9.3 pF to 0.3 pF and a frequency change of 6 MHz. It also illustrates the importance of having a very fast autotuning and matching control system when studying vitrified samples across very large temperature ranges.

With our model and experimental data, we can now estimate the effect of conductivity on the heating rate of water and nanoparticle solutions. Given the dielectric constant, ε′+jε″, and the capacitor dimensions (from the sample cell), we can estimate the capacitance and equivalent series resistance. The actual cell contains two 0.79 mm Teflon® (ε=2.0+j*4.0×10⁻⁴) spacers between the cell and the electrodes as well as two 0.063 mm Kapton® (ε=3.5+j*3.5×10⁻²) acrylate tape films sealing the sample cell. This must be treated as a layered capacitor with each layer calculated separately and added together in series. Due to these additional layers, the voltage across the sample is substantially reduced due to the increased thickness of the capacitor and the dielectric of the layers. Once the capacitance and ESR of each layer is calculated, the power dissipated in each layer can be calculated from the current flowing through the stack of layers, P=Σ i² ESRₙ, where ESR, is the equivalent series resistance of the n^{th} layer. With a deionized water sample, the loaded voltage and current values are similar to those shown in FIG. 11, (Pᵢₙ=10 W, V=990V, and I=10.1 A). However, these values are now slightly lower, because the sample dissipation lowers the Q of the resonator. The estimated current through the sample is ˜121 mA and the voltage drop is only 41 volts. The power delivered to the sample is estimated at 0.015 watt and the measured value is about 0.08-0.1 watt. The calculations indicate that the Kapton® film likely absorbs most of the power since its loss factor is actually quite large, ˜0.05 watt. It seems likely that its power will immediately be transferred into the water, giving a total heating rate of 0.065 watt, which is close to the measured value. While Kapton® has good thermal (cryogenic to high temperature) properties and good electrical insulating properties, it is not ideal for high frequency use. However, it was a film that was capable of sealing the sample cells, and it performed well in this respect even after freeze thaw cycles to LN₂ temperatures. Most of the voltage drop (892V of the 990V across the plates) actually occurs in the Teflon® spacers, and more efficient power coupling would be possible if these were removed and the space closed.

Teaching of theory for ZnO Nanoparticle Heating: The dielectric loss, ε″, is commonly broken into its constituent components: ionic motion, dipole polarization, infrared vibrations, and eventually optical (or plasmon resonance absorption). At RF frequencies, ε″=ε″ₚₒₗₐᵣ+ε_{σ}, where ε″ₚₒₗₐᵣ represents the contribution from molecular dipole, and ε″_{σ} equals the contribution from ionic conductivity, σ. It is a physics problem with Maxwell's equations to show that ε″_{σ}=σ/(ε₀ω), where σ is the electrical conductivity in S/m, ε₀ is the vacuum permittivity constant, and ω is the angular RF frequency. Fortunately, we characterized our ZnO nanoparticles (and DI water) with a conductivity meter. Our DI water had a conductivity of 1×10⁻⁴ S/m, and for comparison, pure water has a conductivity 5.5×10⁻⁶ S/m. Our ZnO nanoparticle solutions tested at: 1 mg/ml=5.5×10⁻⁴ S/m and 10 mg/ml=23×10⁻⁴ S/m. We can calculate that at 50 MHz, ε″_{σ}=0.035 for our DI water, 0.19 for 1 mg/ml ZnO, and 0.81 for 10 mg/ml ZnO. The dielectric properties of saline solutions are known and this is less than 2 ppm salt in the 1 mg/ml sample. Using our model, changing from pure water to our DI water changed the absorbed power from 0.013 to 0.015 watt, a change which is small enough to be in the noise in the current apparatus. The 1 mg/ml ZnO sample heated at 1.9 watts or 1.85 watts after subtracting the sample holder. Computing the ε″_{σ} yields a value of 27, which allows us to back calculate conductivity, σ, of 0.075 S/m. This is 140× the measured conductivity of 5.5×10⁻⁴ S/m. Using the dielectric calculator shows that this requires a salt concentration of 2 parts per thousand. However, this is greater than the concentration of nanoparticles (1 mg/ml). Therefore, our measurements show that the nanoparticles heating with a SAR>20,000 W/g ZnO are correct. The amount of ions in our solution is far too low to be responsible for our heating results. The above detailed discussion is provided to distinguish the present invention from prior art that may have been reported erroneously due to ionic impurities

Heating Mechanism: As taught by the present invention, only the piezoelectric samples heated, and the large heating values obtained are due to a piezoelectric effect of the alternating electric field with the nanoparticle. The nanoparticle is in essence, a nanotransducer. As the electric field enters the nanoparticle, it causes the charges in its crystal lattice to shift, creating a dipole moment that compresses or lengthens the nanoparticle, causing it to vibrate. Without wishing to be bound by theory, this vibration may heat the particle itself, or the vibrations may be transferred to the surround fluid and dissipated as heat. The particle will have resonance frequencies and finding the correct modes or changing the particle size may substantially increase the heating efficiency. This can be measured, with properly suspended (dispersed) particles covering a wide range of sizes. The present method has not previously been reported, and provides new opportunities for nanoparticle heating. Additional heating may come from rotation of the induced dipole in the nanoparticle if it is not aligned with the electric field.

Although the present invention has been described in considerable detail with reference to certain preferred versions thereof, other versions are possible. Therefore, the spirit and scope of the appended claims should not be limited to the description of the preferred versions contained herein, except where required by 35 U.S.C.§ 112 ¶6 or 35 U.S.C.§ 112 (f).

The reader's attention is directed to all references which are filed concurrently with this specification and which are incorporated herein by reference.

All the features in this specification (including any accompanying claims, abstract, and drawings) may be replaced by alternative features serving the same, equivalent or similar purpose, unless expressly stated otherwise. Thus, unless expressly stated otherwise, each feature disclosed in one example only of a generic series of equivalent of similar features. Any element in a claim that does not explicitly state “means for” performing a specified function, or “step for” performing a specific function, is not to be interpreted as a “means” or “step” clause as specified in 35 U.S.C. § 112 ¶ 6 or 35 U.S.C.§ 112 (f). Any element in a claim that does explicitly state “means for” performing a specified function, or “step for” performing a specific function, is to be interpreted as a “means” or “step” clause as specified in 35 U.S.C.§ 112 ¶ 6 or 35 U.S.C.§ 112 (f).

What is claimed is:

1. A method for rapid uniform heating of a material, the method comprising:
(a) providing a target material;
(b) providing a plurality of electrostrictive nanoparticles contained in the target material;
(c) providing a radio frequency alternating electric field that is coupled to the plurality of electrostrictive nanoparticles; and
(d) heating the target material.

2. The method of claim 1, wherein the plurality of electrostrictive nanoparticles are piezoelectric nanoparticles.

3. The method of claim 2, wherein the plurality of electrostricitve nanoparticles are ferroelectric nanoparticles.

4. The method of claim 2, wherein the piezoelectric nanoparticles are either CeO₂ or ZnO nanoparticles.

5. The method of claim 1, wherein the plurality of electrostrictive nanoparticles have an average particle size less than 1 μm as measured by dynamic light scattering.

6. The method of claim 1, wherein the radio frequency alternating electric field is from 1 to 100 MHz.

7. The method of claim 6, wherein the radio frequency alternating electric field is from 30 to 50 MHz.

8. The method of claim 7, wherein the radio frequency alternating electric field strength is 315 kV/m.

9. The method of claim 1, wherein the plurality of electrostrictive nanoparticles have an SAR of at least 1,000 Watts per gram.

10. The method of claim 9, wherein the plurality of electrostrictive nanoparticles have an SAR of at least 18,000 Watts per gram.

11. The method of claim 10, wherein the plurality of electrostrictive nanoparticles have an SAR of at least 20,000 Watts per gram.

12. The method of claim 1, wherein the target material is either: water, living tissue or cryopreserved tissue.

13. The method of claim 1, wherein the plurality of electrostrictive nanoparticles are uniformly dispersed in the target material.

14. The method of claim 1 further comprising the step of:
(e) heating the target material by at least of 1.0° C. per second.

15. The method of claim 1 further comprising the step of:
(e) heating the target material by at least 3.5° C. per second.

16. The method of claim 1, further comprising:
(f) providing a first electrode and a second electrode;
wherein, the target material is placed between the first electrode and the second electrode, wherein the first electrode, the target material and the second electrode are assembled to form a capacitor cell; and
wherein, the step of providing a radio frequency alternating electric field exposes the target material to the radio frequency alternating electric field.

17. The method of claim 16, wherein the capacitor cell further comprises an electrically insulating housing for the target material.

18. The method of claim 17, wherein the electrically insulating housing is made from either Teflon®, Rexolite®, Kapton®, or combinations thereof.
